(19) United States
(12) Patent Application Publication
(10) Pub. No.: US 2007/0155654 A1
(43) Pub. Date: Jul. 5, 2007

(54) Novel formulations

(75) Inventor: Liselotte Langkjaer, Holte (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/701,083

(22) Filed: Feb. 1, 2007

Related U.S. Application Data
(63) Continuation of application No. 10/422,239, filed on Apr. 24, 2003.

(30) Foreign Application Priority Data
May 7, 2002 (DK) PA 2002 00684

(51) Int. Cl. A61K 38/28

(52) U.S. Cl. 514/3

(57) ABSTRACT

Stable, soluble insulin formulations having both a fast and a long action.

### CROSS-REFERENCE TO RELATED APPLICATIONS

This application is a continuation of application Ser. No. 10/422,239 filed on Apr. 24, 2003 and claims priority under 35 U.S.C. 119 of Danish application no. PA 2002 00684 filed May 7, 2002, the contents of which are fully incorporated herein by reference.

### FIELD OF INVENTION

This invention relates to pharmaceutical formulations containing insulin aspart and insulin detemir, wherein insulin detemir has a profile of action which is identical or substantially identical with the profile of action of insulin detemir in the absence of insulin aspart. The invention also relates to methods of treating diabetes which utilize the pharmaceutical formulations of the invention.

### BACKGROUND OF THE INVENTION

Diabetes is a general term for disorders in man having excessive urine excretion as in diabetes mellitus and diabetes insipidus. Diabetes mellitus is a metabolic disorder in which the ability to utilize glucose is partly or completely lost. About 5% of all people suffer from diabetes. Since the introduction of insulin in the 1920's, continuous strides have been made to improve the treatment of diabetes mellitus. To help avoid extreme glycemia levels, diabetic patients often practice multiple daily injection therapy, whereby, for example, fast-acting insulin is administered with each meal and long-acting or intermediate-acting insulin is administered once or twice daily to cover the basal need.

In the treatment of diabetes mellitus, many varieties of insulin formulations have been suggested and used, such as regular insulin, isophane insulin (designated NPH), insulin zinc suspensions (such as Semilente®, Lente®, and Ultralent®), and biphasic isophane insulin. As diabetic patients are treated with insulin for several decades, there is a major need for safe and life quality improving insulin formulations. Some of the commercially available insulin formulations are characterized by a fast onset of action and other formulations have a relatively slow onset but show a more or less prolonged action. Fast-acting insulin formulations are usually solutions of insulin, while retarded acting insulin formulations can be suspensions containing insulin in crystalline and/or amorphous form precipitated by addition of zinc salts alone or by addition of protamine or by a combination of both. In addition, some patients are using formulations having both a fast onset of action and a more prolonged action. Such a formulation may be an insulin solution wherein protamine insulin crystals are suspended. Some patients do themselves prepare the final formulation by mixing a fast acting insulin solution with a protracted acting insulin suspension formulation in the ratio desired by the patient in question.

Human insulin consists of two polypeptide chains, the so-called A and B chains which contain 21 and 30 amino acid residues, respectively. The A and B chains are interconnected by two cystine disulphide bridges. Insulin from most other species has a similar construction, but may not contain the same amino acid residues at the same positions.

The development of the process known as genetic engineering has made it possible to prepare a great variety of insulin compounds being analogous to human insulin. In these insulin analogues, one or more of the amino acids have been substituted with other amino acids which can be coded for by the nucleotide sequences.

Normally, insulin formulations are administered by subcutaneous injection. What is important for the patient, is the action profile of the insulin formulation which is the action of insulin on the glucose metabolism as a function of the time from the injection. In this profile, inter alia, the time for the onset, the maximum value, and the total duration of action are important. A variety of insulin formulations with different action profiles are desired and requested by the patients. One patient may, on the same day, use insulin formulations with very different action profiles. The action profile requested is, for example, depending on the time of the day and the amount and composition of any meal eaten by the patient.

There is a big need for insulin formulations with different profiles of release of insulin. A patient may, during the day, use insulin formulations with different profiles of release. For example, the patient may, before a meal, use a fast-acting insulin formulation with no retarded action. Another patient may, before a meal, use a formulation having both a fast action and a retarded action. In such a formulation having both a fast action and a retarded action, the ratio between fast action and retarded action may vary considerably. Before a patient goes to sleep, the patient may use a long-acting insulin formulation. Some patients will, before they go to sleep, use a formulation having both a fast action and a retarded action.

### SUMMARY OF THE INVENTION

One object of the present invention is to furnish insulin formulations having a convenient profile of action.

Another object of the present invention is to furnish soluble insulin formulations having both a fast onset of action and also a retarded action.

Another object of the present invention is to furnish insulin formulations having no or only a minor amount of non-dissolved material.

Another object of the present invention is to furnish insulin formulations containing both a fast and long acting insulin component wherein the two insulin components acts as or acts substantially as they would have acted if they had been the only insulin components present in the formulation.

Another object of the present invention is to furnish insulin formulations having a profile of release which is very predictable, both from time to time an also form patient to patient.

### DESCRIPTION OF THE INVENTION

It has surprisingly been found that aqueous insulin formulations comprising about 15-85% (on a unit to unit basis) of insulin aspart and the remaining part of insulin activity originating from insulin detemir, give profiles of release which are convenient for different patient groups. The systematic chemical names of insulin aspart and insulin detemir are Asp^{B28} human insulin and Lys^{B29}(N^{ε}-tetradecanoyl) des(B30) human insulin, respectively. Collectively they are herein referred to as the insulin components.

The formulations of the inventions have no or only a minor content of non-dissolved material. Furthermore, in the formulations of this invention, the two insulin components act as or act substantially as they would have acted if they had been the only insulin components present. The formulations of the present invention have a profile of release which is very predictable, both from time to time and also from patient to patient.

The pharmaceutical formulation of this invention may be prepared using the conventional techniques of the pharmaceutical industry which involves dissolving and mixing the pertinent ingredients as appropriate to give the desired end product.

Thus, according to one procedure, on one hand, insulin aspart and, on the other hand, insulin detemir is dissolved in an amount of water, the total volume of which is somewhat less than the final volume of the formulation to be prepared. An isotonic agent, a preservative, and, optionally, a buffer is added as required and the pH value of the solution is adjusted—if necessary—using an acid, for example, hydrochloric acid, or a base, for example, aqueous sodium hydroxide as needed. Finally, the volume of the solution is adjusted with water to give the desired concentration of the ingredients.

In a preferred embodiment of this invention, the formulation contains an agent rendering the solution isotonic, an antimicrobial preservative, a pH-buffering agent, and a suitable zinc salt.

In a preferred embodiment of this invention, the formulation has a total amount of the insulin in the range from about 10 U/ml to about 1500 U/ml, preferably in the range from about 40 U/ml to about 1000 U/ml, more preferred in the range from about 100 U/ml to about 500 U/ml, for example, 100, 200, 400, or 500 U/ml. The term “U”, when used herein, refers to insulin units. For insulin aspart, one unit equals 6 nmol (about 40 μg) and for insulin detemir, one unit equals 24 nmol (about 160 μg).

In a preferred embodiment of this invention, the preservative is phenol, m-cresol or a mixture of phenol and m-cresol. In a further preferred embodiment of this invention, the total concentration of phenol and/or m-cresol is in the range from about 20 mM to about 50 mM, preferably in the range from about 30 mM to about 45 mM. The concentration of phenol and/or m-cresol is, inter alia, dependent on the concentration of insulin.

In a preferred embodiment of this invention, the formulation has a content of zinc ions at the disposal of insulin in proportions in the range from about 2.3 to about 4.5 Zn²⁺ per hexamer insulin (corresponding to from about 0.38 to about 0.75 Zn²⁺/monomer insulin) where it is understood that the content of zinc is expressed per insulin hexamer as a theoretical value, i.e., as the number of zinc atoms per 6 molecules of monomeric insulin, independent of whether all insulin actually is present as hexameric insulin or not. The zinc salt used for preparing the formulations of this invention may, for example, be zinc chloride, zinc oxide or zinc acetate.

In a preferred embodiment of this invention, the isotonic agent is glycerol, mannitol, sorbitol or a mixture thereof at a concentration in the range from about 100 to 250 mM.

In another preferred embodiment of this invention, the formulation contains halogenide ions, preferably as sodium chloride, in an amount corresponding to from about 1 mM to about 100 mM, preferably from about 5 mM to about 40 mM.

In a preferred embodiment of this invention, the pH buffer is sodium phosphate, TRIS (trometamol), N-glycylglycine or L-arginine. Preferably, the pH buffer is a physiologically acceptable buffer in a concentration in the range from about 3 mM to about 20 mM, preferably from about 5 mM to about 15 mM. In a preferred embodiment of this invention, the formulations of this invention have a pH value in the range from about 7.0 to about 8.0.

In a preferred embodiment of this invention, the formulation of this invention has a content of non-dissolved material below about 0.1%, preferably below 0.01% (weight per weight).

Administration of the formulations of this invention may be via any route known to be effective by the physician of ordinary skill. Parenteral and preferably subcutaneous administration is preferred.

The amount of the formulation of this invention that is administered to treat diabetes depends on a number of factors, among which are included the patient's sex, weight, physical activity, and age, diet of the patient, the underlying causes of the condition or disease to be treated, the route of administration and bioavailability, the persistence of the administered insulin or insulin analogues in the body, the specific formulation used, the potency of the insulin or insulin analogue used, a possible combination with other drugs, the severity of the case of diabetes, and the interval between dosages, if any interval. It is within the skill of the ordinary physician to titrate the dose and frequency of administration of the formulation of this invention to achieve the desired result. It is recommended that the daily dosage of the insulin components used in the formulation according to this invention be determined for each individual patient by those skilled in the art in a similar way as for known insulin compositions.

This invention is further illustrated by the following examples which, however, are not to be construed as limiting the scope of protection. The features disclosed in the foregoing description and in the following examples may, both separately and in any combination thereof, be material for realizing this invention in diverse forms thereof.

### EXAMPLE 1

67 U Insulin Per ml Containing 50% (U/U) Insulin Aspart and 50% (U/U) Insulin Detemir

A solution with the following composition was prepared: Insulin aspart 33.3 U/ml (200 nmol/ml), Insulin detemir 33.3 U/ml (800 nmol/ml), phenol 1.50 mg/ml (16 mM), m-cresol 1.72 mg/ml (16 mM), mannitol 30 mg/ml (165 mM), dibasic sodium phosphate dihydrate 1.25 mg/ml (7 mM), sodium chloride 1.75 mg/ml (30 mM), zinc chloride and zinc acetate up to a total concentration of 32.7 μg Zn²⁺/ml (3 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml using aseptic technique.

The blood glucose profile of the formulation after subcutaneous injection was tested in a cross over study in fasted pigs and compared with the profile after separate, simultaneous injections of Insulin Aspart (example 8) and Insulin Detemir (example 9) in the same doses.

[image]

### EXAMPLE 2

100 U Insulin Per ml Containing 85% (U/U) Insulin Aspart and 15% (U/U) Insulin Detemir

A solution with the following composition was prepared: Insulin aspart 85 U/ml (510 nmol/ml), Insulin detemir 15 U/ml (360 nmol/ml), phenol 1.80 mg/ml (19 mM), m-cresol 2.06 mg/ml (19 mM), glycerol 16 mg/ml (174 mM), dibasic sodium phosphate dihydrate 0.9 mg/ml (5 mM), sodium chloride 1.2 mg/ml (20 mM), zinc chloride and zinc acetate up to a total concentration of 28.4 μg Zn²⁺/ml (3.0 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml using aseptic technique.

### EXAMPLE 3

100 U Insulin Per ml Containing 70% (U/U) Insulin Aspart and 30% (U/U) Insulin Detemir

A solution with the following composition was prepared: Insulin aspart 70 U/ml (420 nmol/ml), Insulin detemir 30 U/ml (720 nmol/ml), phenol 1.80 mg/ml (19 mM), m-cresol 2.06 mg/ml (19 mM), glycerol 16 mg/ml (174 mM), dibasic sodium phosphate dihydrate 0.9 mg/ml (5 mM), sodium chloride 1.2 mg/ml (20 mM), zinc chloride and zinc acetate up to a total concentration of 31.1 μg Zn²⁺/ml (2.5 Zn²⁺/hexamer). Hydrochloric acid and sodium hydroxide were used for dissolution of the insulin and adjustment of pH to 7.20. Finally the solution was sterilized by filtration and filled into sterile Penfill® cartridges 1.5 ml or 3 ml as well as vials 2 ml using aseptic technique.

### EXAMPLE 4

100 U Insulin Per ml Containing 50% (U/U) Insulin Aspart and 50% (U/U) Insulin Detemir

A solution with the following composition was prepared: Insulin aspart 50 U/ml (300 nmol/ml), Insulin detemir 50 U/ml (1200 nmol/ml), phenol 1.80 mg/ml (19 mM), m-cresol 2.06 mg/ml (19 mM), glycerol 16 mg/ml (174 mM), dibasic sodium phosphate dihydrate 0.9 mg/ml (5 mM), sodium chloride 1.2 mg/ml (20 mM), zinc chloride and zinc acetate up to a total concentration of 49 μg Zn²⁺/ml (3.0 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml or 3 ml as well as vials 2 ml using aseptic technique.

### EXAMPLE 5

100 U Insulin Per ml Containing 30% (U/U) Insulin Aspart and 70% (U/U) Insulin Detemir

A solution with the following composition was prepared: Insulin aspart 30 U/ml (180 nmol/ml), Insulin detemir 70 U/ml (1680 nmol/ml), phenol 1.80 mg/ml (19 mM), m-cresol 2.06 mg/ml (19 mM), glycerol 16 mg/ml (174 mM), dibasic sodium phosphate dihydrate 0.9 mg/ml (5 mM), sodium chloride 1.2 mg/ml (20 mM), zinc chloride and zinc acetate up to a total concentration of 60.8 μg Zn²⁺/ml (3.0 Zn²⁺/hexamer). Hydrochloric acid and sodium hydroxide were used for dissolution of the insulin and adjustment of pH to 7.60. Finally the solution was sterilized by filtration and filled into sterile Penfill® cartridges 1.5 ml or 3 ml as well as vials 2 ml using aseptic technique.

### EXAMPLE 6

100 U Insulin Per ml Containing 15% (U/U) Insulin Aspart and 85% (U/U) Insulin Detemir

A solution with the following composition was prepared: Insulin aspart 15 U/ml (90 nmol/ml), Insulin detemir 85 U/ml (2040 nmol/ml), phenol 1.80 mg/ml (19 mM), m-cresol 2.06 mg/ml (19 mM), glycerol 16 mg/ml (174 mM), dibasic sodium phosphate dihydrate 0.9 mg/ml (5 mM), sodium chloride 1.2 mg/ml (20 mM), zinc chloride and zinc acetate up to a total concentration of 69.6 μg Zn²⁺/ml (3.0 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml using aseptic technique.

### EXAMPLE 7

100 U Insulin Per ml Containing 50% (U/U) Insulin Aspart and 50% (U/U) Insulin Detemir

A solution with the following composition was prepared: Insulin aspart 50 U/ml (300 nmol/ml), Insulin detemir 50 U/ml (1200 nmol/ml), phenol 1.80 mg/ml (19 mM), m-cresol 2.06 mg/ml (19 mM), mannitol 30 mg/ml (165 mM), dibasic sodium phosphate dihydrate 0.9 mg/ml (5 mM), sodium chloride 1.2 mg/ml (20 mM), zinc chloride and zinc acetate up to a total concentration of 49 μg Zn²⁺/ml (3.0 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml or 3 ml as well as vials 2 ml using aseptic technique.

### EXAMPLE 8

Insulin Aspart 600 nmol/ml (Reference)

A solution with the following composition was prepared: Insulin aspart 100 U/ml (600 nmol/ml), phenol 1.50 mg/ml (16 mM), m-cresol 1.72 mg/ml (16 mM), glycerol 16 mg/ml (174 mM), dibasic sodium phosphate dihydrate 1.25 mg/ml (7 mM), sodium chloride 1.75 mg/ml (30 mM), zinc chloride up to a total concentration of 19.6 μg Zn²⁺/ml (3.0 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml using aseptic technique.

### EXAMPLE 9

Insulin Detemir 1200 nmol/ml (Reference)

A solution with the following composition was prepared: Insulin detemir 50 U/ml (1200 nmol/ml), phenol 1.50 mg/ml (16 mM), m-cresol 1.72 mg/ml (16 mM), glycerol 16 mg/ml (174 mM), dibasic sodium phosphate dihydrate 1.25 mg/ml (7 mM), sodium chloride 1.75 mg/ml (30 mM), zinc chloride up to a total concentration of 39.2 μg Zn²⁺/ml (3.0 Zn²⁺/hexamer). Hydrochloric acid and sodium hydroxide were used for dissolution of the insulin and adjustment of pH to 7.40. Finally the solution was sterilized by filtration and filled into sterile Penfill® cartridges 1.5 ml using aseptic technique.

1. A pharmaceutical formulation comprising insulin aspart and insulin detemir, wherein the ratio between insulin aspart and insulin detemir is in the range from 15:85 to 85:15, on a unit (U) to unit (U) basis.

2. The formulation according to claim 1, said formulation further comprising an isotonicity agent, an antimicrobial preservative, a pH-buffering agent, and a suitable zinc salt.

3. The formulation according to claim 2, wherein the formulation has a pH value from about 7 to about 8.

4. The formulation according to claim 1, wherein the insulin is present in a concentration of from about 10 U/ml to about 1500 U/ml.

5. The formulation according to claim 1, wherein the insulin is present in a concentration of from about 40 U/ml to about 1000 U/ml.

6. The formulation according to claim 1, wherein the insulin is present in a concentration of from about 100 U/ml to about 500 U/ml.

7. The formulation according to claim 2, wherein the preservative is phenol, m-cresol or a mixture of phenol and m-cresol.

8. The formulation according to claim 7, wherein the phenol and/or m-cresol is present in a total concentration of from about 20 mM to about 50 mM.

9. The formulation according to claim 7, wherein the phenol and/or m-cresol is present in a total concentration of from about 30 mM to about 45 mM.

10. The formulation according to claim 2, wherein said formulation contains from about 2.3 to about 4.5 Zn²⁺ per insulin hexamer.

11. The formulation according to claim 2, wherein the zinc salt is zinc chloride, zinc oxide or zinc acetate.

12. The formulation according to claim 2, wherein said formulation further contains halogenide ions.

13. The formulation according to claim 12, wherein the halogenide ion is sodium chloride in a concentration of from about 1 to about 100 mM.

14. The formulation according to claim 12, wherein the halogenide ion is sodium chloride in a concentration of from about 5 to about 40 mM.

15. The formulation according to claim 2, wherein the isotonicity agent is glycerol, mannitol, sorbitol, or a mixture thereof in a concentration in a concentration range of from about 100 to about 250 mM.

16. The formulation according to claim 2, wherein the pH-buffer is sodium phosphate, TRIS (trometamol), N-glycylglycine, or L-arginine.

17. The formulation, according to claim 16, wherein the pH-buffer is a physiologically acceptable buffer in a concentration of from about 3 mM to about 20 mM.

18. The formulation, according to claim 16, wherein the pH-buffer is a physiologically acceptable buffer in a concentration of from about 5 mM to about 15 mM.

19. A method of treating diabetes in a patient in need of such treatment, said method comprising administering to said patient a therapeutically effective amount of a pharmaceutical formulation according to claim 1.
